(19) United States
(12) Patent Application Publication
(10) Pub. No.: US 2005/0137222 A1
(43) Pub. Date: Jun. 23, 2005

(54) Treatment of insomnia in human patients

(75) Inventors: Bjarke Ebert, Farum (DK); Jonas Lundahl, Hollviken (SE)

Correspondence Address: DARBY & DARBY P.C., P. O. BOX 5257, NEW YORK, NY, 10150-5257, US

(73) Assignee: H. Lundbeck A/S, Valby-Copenhagen (DK)

(21) Appl. No.: 11/014,019

(22) Filed: Dec. 15, 2004

Related U.S. Application Data
(60) Provisional application No. 60/531,046, filed on Dec. 18, 2003.

(51) Int. Cl. A61K 31/4745

(52) U.S. Cl. 514/302

(57) ABSTRACT

The present invention concerns a method for treating insomnia in a human subject in need thereof by administering to said subject an amount of gaboxadol per day, said amount being effective for the treatment of insomnia. In various preferred embodiments, the effective amount is from 5 to 15 mg gaboxadol in oral dosage form, administered from one hour before up until bedtime.

This application claims the benefit of U.S. Provisional Application No. 60/531,046 filed Dec. 18, 2003, which is incorporated herein by reference in its entirety.

### FIELD OF INVENTION

The present invention relates to the use of gaboxadol for the preparation of medicaments useful for the treatment of insomnia in a human subject, a method for treating insomnia in a human subject, and a pharmaceutical composition comprising an amount of gaboxadol effective for the treatment of insomnia.

### BACKGROUND OF THE INVENTION

Gaboxadol (THIP or 4,5,6,7-tetrahydroisoxazolo(5,4-c)pyridin-3-ol) described in EP patent 0000338 B1, and in EP Patent 0840601 B1 has shown great potential in the treatment of sleep disorders in general.

### DESCRIPTION OF THE INVENTION

Several studies in animals have indicated the usefulness of gaboxadol as an agent for increasing total amount of nonREMS and lengthening the duration of the nonREMS and REMS episodes. Studies in healthy human subjects have indicated that gaboxadol is able to increase sleep maintenance and to promote deep sleep, while having no disrupting influence on REM sleep.

The present inventors are the first to realize the usefulness of gaboxadol as an oral treatment of insomnia in human subjects. Accordingly, no treatment regimens have been proposed for treatment of insomnia in human subjects, no effects of cessation of treatment in human subjects have been studied, and no abuse or dependency of treatment in human subjects have been investigated.

We have now shown that gaboxadol is an effective treatment for insomnia in human subjects, in particular primary insomnia.

Additionally, gaboxadol has shown a significant improved effect in human subjects suffering from impaired day time functioning, in particular such subjects suffering from primary insomnia. Impaired day time functioning may be due to suffering from a sleep disorder or any disease, disorder or condition leading to deprivation of sleep, or obstruction of sleep, such as primary insomnia, secondary insomnia, circadian rhythm sleep disorders, sleep disorders due to occasional stress, sleep apnea, narcolepsy, sleep paralysis, snoring, night terrors, night sweats, delayed sleep phase syndrome, depression, chronic tension type headache, fibromyalgia, neuropathic pain, chronic pain, alcohol abuse, or arthritis.

The objective of the invention is to provide an effective treatment of insomnia in a human subject, in particular primary insomnia and insomnia secondary to another mental disorder, a general medical condition or induced by a substance (secondary insomnia).

A further objective of the invention is to provide an effective treatment of insomnia in a human subject, without causing rebound insomnia upon cessation of treatment, in particular long-term treatment.

A further objective of the invention is to provide an effective treatment of insomnia in a human subject, without causing abuse or dependency of treatment, in particular long-term treatment. In this respect the studies performed revealed that it was particularly advantageous to administer gaboxadol at bedtime, such as from about 1 hour, from about 45 minutes, from about ½ hour, from about fifteen minutes, or from about 5 minutes, before going to sleep.

Further objectives of the invention will become apparent upon reading the present specification.

Gaboxadol has the general formula 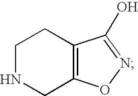
and throughout the description “gaboxadol” is intended to include any form of the compound, such as the base (zwitter ion), pharmaceutically acceptable salts, e.g. pharmaceutically acceptable acid addition salts, hydrates or solvates of the base or salt, as well as anhydrates, and also amorphous, or crystalline forms.
Definitions

The term “insomnia” is intended to mean a disorder characterized by abnormalities in the amount, quality and timing of sleep and includes primary insomnia and insomnia secondary to another mental disorder, a general medical condition, or induced by a substance (hereafter referred to as secondary insomnia). Examples of secondary insomnia include but are not limited to insomnia accompanying a circadian rhythm sleep disorder, or a sleep disorder due to occasional stress.

Primary insomnia is associated with complaint in initiating or maintaining, or nonrestorative sleep not exclusively occurring due to another mental disorder, physiological effects of a substance or a general medical condition. Secondary insomnia is associated with complaint in initiating or maintaining, or nonrestorative sleep occurring due to another mental disorder, physiological effects of a substance or a general medical condition. Such a substance may be, e.g., an acetylcholinesterase inhibitor or a beta blocker.

The treatment is typically given for a duration of less than a week (short term treatment), from 1 to 4 weeks (intermediate term treatment) or for a period exceeding 4 weeks (long-term treatment). A special type of long-term treatment is chronic treatment.

The term “elderly” is intended to mean humans from 65 years and above.

The term “adults” is intended to mean humans from 18 to 64 years.

The term “children” is intended to mean humans from 0 to 17 years.

According to the present invention an effective medicament with no significant side-effects for the treatment of insomnia in human subjects is provided.

In one aspect, the present invention relates to a pharmaceutical composition comprising an amount of gaboxadol effective for the treatment of insomnia. Individual embodiments are primary insomnia and secondary insomnia. The treatment is typically given during less than a week, from 1 week up to 4 weeks, or for a period exceeding 4 weeks. The pharmaceutical composition is an oral dose form, such as a solid oral dose form, typically tablets or capsules, or a liquid oral dose form, such as a solution. In a particular embodiment the pharmaceutical composition is a solid oral dose form, typically tablets or capsules.

In a further embodiment, gaboxadol is selected from the zwitter ion, typically a hydrate thereof, although the anhydrate is also suitable. A typical embodiment is the zwitter ion monohydrate.

In a further embodiment, gaboxadol is selected from an acid addition salt, typically a pharmaceutically acceptable acid addition salt. A typical embodiment is an organic acid addition salt, such as any one of the maleic, fumaric, benzoic, ascorbic, succinic, oxalic, bis-methylensalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, p-amino-benzoic, glutamic, benzene sulfonic or theophylline acetic acid addition salts. Another typical embodiment is an inorganic acid addition salt, such as any one of the hydrochloric, hydrobromic, sulfuric, sulfamic, phosphoric or nitric acid addition salts.

Typically, gaboxadol is in the form of the hydrochloric acid salt, the hydrobromic acid salt, or the zwitter ion monohydrate.

In a further embodiment gaboxadol is crystalline, such as the crystalline hydrochloric acid salt, the crystalline hydrobromic acid salt, or the crystalline zwitter ion monohydrate. In a further embodiment, the amount of gaboxadol in a composition ranges from 2.5 mg to 20 mg, such as 5 mg to 15 mg. The amount of gaboxadol is calculated based on the free base form. In typical embodiments, the amount of gaboxadol in the composition is 2.5 mg, 5 mg, 7.5 mg, 10 mg, 12.5 mg, 15 mg, 17.5 mg or 20 mg. Other amounts of gaboxadol in this range may be used, such as but not limited to: 3 mg, 4 mg, 6 mg, 7 mg, 8 mg, 9 mg, 11 mg, 12 mg, 13 mg, 14 mg, 16 mg, 17 mg, 18 mg or 19 mg.

In a further aspect, the present invention relates to a method for treating insomnia in a human subject in need thereof comprising administering to said subject an amount of gaboxadol per day, said amount being effective for the treatment of insomnia. In typical embodiments, the amount of gaboxadol administered per day is 2.5 mg, 5 mg, 7.5 mg, 10 mg, 12.5 mg, 15 mg, 17.5 mg or 20 mg. Other amounts of gaboxadol in this range may be used, such as but not limited to: 3 mg, 4 mg, 6 mg, 7 mg, 8 mg, 9 mg, 11 mg, 12 mg, 13 mg, 14 mg, 16 mg, 17 mg, 18 mg or 19 mg.

Typical embodiments of gaboxadol are selected from an acid addition salt, such as a pharmaceutically acceptable acid addition salt, e.g. selected from the hydrochloride or hydrobromide salt; or a zwitter ion hydrate, such as the zwitter ion monohydrate; or the zwitter ion anhydrate.

Typical embodiments of the effective amount of gaboxadol range from about 2.5 mg to about 20 mg, such as 5 mg to 15 mg, of gaboxadol per day. Most conveniently, gaboxadol is in a crystalline form.

Typical embodiments of insomnia are selected from subjects with primary insomnia or secondary insomnia.

The subject may be any subject suffering from insomnia, and is selected from children, adults, or elderly.

Gaboxadol is administered as an oral dose form, such as a solid oral dose form, typically tablets or capsules, or as a liquid oral dose form. Gaboxadol is most conveniently administered orally in unit dosage forms such as tablets or capsules, containing the active ingredient in an amount from about 2.5 to about 20 mg, e.g. from about 5 to about 15 mg.

Subjects requiring short-term treatment are typically subjects suffering from circadian rhythm sleep disorders or sleep disorders due to occasional stress.

Subjects requiring intermediate, long-term or chronic treatment are typically subjects suffering from primary insomnia or secondary insomnia.

In a further aspect, the present invention relates to use of gaboxadol for preparing a medicament for treating insomnia in a human subject. In particular the medicament comprises an amount of gaboxadol being effective for the treatment of insomnia.

In a certain aspect, the present invention relates to use of gaboxadol for preparing a medicament for treating primary insomnia in an adult or elderly human subject, wherein said medicament comprises an oral dose form comprising 5 to 15 mg of gaboxadol to be administered once daily from about 1 hour before bedtime up until bedtime. Preferably gaboxadol is administered from about ½ hour to 45 minutes before bedtime, up to bedtime, such as from 0 to 45 minutes before bedtime, e.g. from 0 to 30 minutes before bedtime, or from 5 to 20 minutes before bedtime. Any one of the embodiments of gaboxadol may be used. Moreover, the treatment may be intermediate, long-term or chronic treatment.

In particular embodiments, gaboxadol is in the form of an acid addition salt, or a zwitter ion hydrate or zwitter ion anhydrate. In further embodiments, gaboxadol is in the form of the pharmaceutically acceptable acid addition salt selected from the hydrochloride or hydrobromide salt, or in the form of the zwitter ion monohydrate.

Preferably, the effective amount ranges from 2.5 mg to 20 mg of gaboxadol, calculated as the base. Preferably, the gaboxadol is in a crystalline form. Further embodiments of the medicament comprise an effective amount of gaboxadol from 2.5 mg to 20 mg, such as 5 mg to 15 mg, e.g. 2.5 mg, S mg, 7.5 mg, 10 mg, 12.5 mg, or 15 mg. A typical embodiment being 5 mg to 15 mg of crystalline gaboxadol, such as the hydrochloride of gaboxadol. A typical embodiment is use of gaboxadol as the hydrochloride for preparing a medicament comprising an effective amount of gaboxadol from 5 mg to 15 mg, for treating insomnia in a human subject.

The human subject to be treated with gaboxadol may in fact be any subject of the human population, male or female, which may be divided into children, adults, or elderly. Any one of these subject groups relates to an embodiment, thus, a typical embodiment is use of gaboxadol for preparing a medicament comprising an effective amount of gaboxadol, from 2.5 mg to 20 mg, for treating insomnia, such as primary or secondary insomnia, in an elderly human subject.

In a further embodiment, the medicament is an oral dose form. Typically, the medicament is a solid oral dose form, such as tablets or capsules, or a liquid oral dose form. Thus, a typical embodiment is use of gaboxadol for preparing a medicament in an oral dose form comprising an effective amount of the gaboxadol from 2.5 mg to 20 mg, for treating insomnia, such as primary or secondary insomnia, in a human subject, such as an elderly human subject.

In a further embodiment, the treatment is short-term treatment. In a further embodiment the treatment is intermediate term treatment. In a further embodiment the treatment is long term treatment. In a further embodiment the treatment is chronic treatment. A typical embodiment is use of gaboxadol for preparing a medicament, such as in an oral dose form, comprising an effective amount of the gaboxadol from 2.5 mg to 20 mg, for long term treatment of insomnia, such as primary or secondary insomnia, in a human subject, such as an elderly human subject.

In a certain aspect, the present invention relates to a method for treating primary insomnia in an adult or elderly human subject in need thereof comprising administering to said subject an oral dose form comprising 5 to 15 mg of gaboxadol per day wherein gaboxadol is administered from about 1 hour before bedtime up until bedtime. Preferably gaboxadol is administered from about ½ hour to 45 minutes before bedtime up until bedtime, such as from 0 to 45 minutes before bedtime, e.g. from 0 to 30 minutes before bedtime, or from 5 to 20 minutes before bedtime. Any one of the embodiments of gaboxadol may be used. Moreover, the treatment may be intermediate, long-term or chronic treatment.

In a further aspect the present invention relates to a method of improving day time functioning in a human subject in need thereof comprising administering to said subject an oral dose form comprising 5 to 15 mg of gaboxadol per day. In a still further aspect the present invention relates to a method of treating impaired day time functioning in a human subject in need thereof comprising administering to said subject an oral dose form comprising 5 to 15 mg of gaboxadol per day. Such human subject may be suffering from a sleep disorder or any disease, disorder or condition leading to deprivation of sleep, or obstruction of sleep, such as primary insomnia, secondary insomnia, circadian rhythm sleep disorders, sleep disorders due to occasional stress, sleep apnea, narcolepsy, sleep paralysis, snoring, night terrors, night sweats, delayed sleep phase syndrome, depression, chronic tension type headache, fibromyalgia, neuropathic pain, chronic pain, alcohol abuse, or arthritis. Each of these disorders, diseases, or conditions may be the subject of one or more claims, for instance, the invention concerns in one embodiment a method of improving day time functioning in a human subject suffering from primary insomnia in need thereof comprising administering to said subject an oral dose form comprising 5 to 15 mg of gaboxadol per day.

According to the invention gaboxadol may be used as the base (i.e. the zwitter ion) or as a pharmaceutically acceptable acid addition salt thereof or as an anhydrate or hydrate or solvate of such salt or base. The salts of the compound used in the invention are salts formed with non-toxic organic or inorganic acids. Exemplary of such organic salts are those with maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, p-amino-benzoic, glutamic, benzene sulfonic and theophylline acetic acids, as well as the 8-halotheophyllines, for example 8-bromo-theophylline. Exemplary of such inorganic salts are those with hydrochloric, hydrobromic, sulfuric, sulfamic, phosphoric and nitric acids. Gaboxadol may also be used as the zwitter ion, e.g. the mono hydrate thereof.

The acid addition salts according to the invention may be obtained by treatment of gaboxadol with the acid in an inert solvent followed by precipitation, isolation and optionally re-crystallisation by known methods and if desired micronization of the crystalline product by wet or dry milling or another convenient process, or preparation of particles from a solvent-emulsification process. Suitable methods are described in EP patent 0000338.

Precipitation of the salt is typically carried out in an inert solvent, e.g. an inert polar solvent such as an alcohol (e.g. ethanol, 2-propanol and n-propanol), but water or mixtures of water and inert solvent may also be used.

According to the invention, gaboxadol should be administered orally, and it may be presented in any suitable form for such administration, e.g. in the form of tablets, capsules, powders, syrups or solutions. Preferably, and in accordance with the purpose of the present invention, gaboxadol is administered in the form of a solid pharmaceutical entity, suitably as a tablet or a capsule.

Methods for the preparation of solid or liquid pharmaceutical preparations are well known in the art. See e.g., Remington's Pharmaceutical Sciences, 20th edition, Mack Publishing Company, 2000. Tablets may thus be prepared by mixing the active ingredients with an ordinary carrier, such as an adjuvant and/or diluent, and subsequently compressing the mixture in a convenient tabletting machine. Examples of adjuvants and/or diluents comprise: corn starch, lactose, talcum, magnesium stearate, gelatine, lactose, gums, and the like. Any other adjuvant or additive such as colourings, aroma, preservatives, etc. may also be used provided that they are compatible with the active ingredients. The pharmaceutical compositions of the invention thus typically comprise an effective amount of gaboxadol and a pharmaceutically acceptable carrier.

A suitable formulation of gaboxadol is described in WO 02/094225 filed May 17, 2002, published Nov. 28, 2002. Without limiting the invention in any way, it is intended that any one of the aspects or embodiments of this patent application is suitable for the medicaments or pharmaceutical compositions herein. For example, WO 02/094225 entitled “Granuar Preparations of Gaboxadol” relates to a specific melt granulation which is particularly useful for formulation of an acid addition salt, but the present invention is in no way limited to such a formulation.

Experimental Procedure

Doses are 2.5 to 20 mg per day as previously stated. The doses are not different between the subject diagnoses or treatment durations.

Effect is confirmed for short-term treatment in studies using acute treatment in healthy subjects exposed to a phase advance model. Assessment of effect on insomnia during intermediate term treatment duration is performed in primary insomnia subjects using treatment duration up to 4 weeks. Long term or chronic treatment is assessed in 12 month safety studies. Subject populations include primary insomnia subjects with age range 18-65 yrs (adults) and 65 and above (elderly).

Abuse and dependency is evaluated using a clinical abuse liability study as well as monitoring subjects in the withdrawal period after gaboxadol.

Results

Two studies in adult subjects with primary insomnia and in healthy subjects using a phase advance model have demonstrated that gaboxadol improves sleep maintenance as measured by polysomnograph (PSG). Sleep maintenance is measured by either of parameters wakefulness after sleep onset, number of awakenings after sleep onset or total sleep time. Data from the clinical study including a phase advance model in healthy subjects also demonstrated that subjects perceive this effect as decreased wakefulness. The effect was observed at doses as low as about 5 mg per day. Furthermore, gaboxadol facilitated sleep initiation in studies conducted using primary insomnia subjects and subjects in a phase advance model. In these studies, the effect was also confirmed by the subjects own feelings of the sleep initiation. Based both on PSG and on subjective diary recording, gaboxadol doses of 5 to 15 mg are highly preferred to achieve effect in adults and elderly (gaboxadol was administered from ½ hour before bedtime up to immediately before going to bed).

Moreover, gaboxadol induced a dose-dependent increase in duration of slow wave sleep (SWS), that is, stages 3 and 4, which is considered the deepest stage of sleep. The effect ranged between +15 and 46% compared to placebo. The enhancement of SWS observed in the EEG was identified in the spectral analysis as an increase of slow wave activity with similar effect range as described above for slow wave sleep.

Improvement in Daytime Function

The daytime performance (used interchangeably with daytime function) in adult (18-65 yrs) primary insomnia subjects (Diagnostic and Statistical Manual of Mental Disorders—Fourth Edition, DSM WV-TR) has been assessed in a 3 week placebo controlled parallel group, outpatient study. The performance was measured with standardized questions using a 100 point Visual Analogue Scale (VAS). The questions asked for subjects to report on their daytime ability to function, tiredness, energy and relaxedness. Subject responses were recorded in a diary in the evening on the day after each treatment night (gaboxadol was administered about ½ hour before bedtime). Moreover, daytime function was recorded once weekly using the Sheehan disability scale (Sheehan et al., 1996, “The measurement of disability,” International Clinical Psychopharmacology 11 (suppl. 3), 89-95).

Based on weekly means of above questions and scale it may be concluded that gaboxadol in doses 5, 10 and 15 mg significantly improve daytime performance. The effect is more pronounced during week 2 and 3.

Throughout this application various references are cited; the contents of each cited reference is incorporated herein by reference in its entirety for all purposes.

1. A method for treating insomnia in a human subject in need thereof comprising administering to said subject an amount of gaboxadol per day, the amount being effective for the treatment of insomnia.

2. The method of claim 1, wherein gaboxadol is administered in an oral dose form.

3. The method of claim 1, wherein gaboxadol is in the form of an acid addition salt, a zwitter ion hydrate, or a zwitter ion anhydrate.

4. The method of claim 3, wherein the acid addition salt is selected from the group consisting of a hydrochloride salt and a hydrobromide salt.

5. The method of claim 3, wherein the zwitter ion hydrate is a zwitter ion monohydrate.

6. The method of claim 1, wherein the amount of gaboxadol per day ranges from 2.5 mg to 20 mg.

7. The method of claim 1, wherein insomnia is selected from the group consisting of primary insomnia and secondary insomnia.

8. The method of claim 7, wherein secondary insomnia is selected from the group consisting of a circadian rhythm sleep disorder and a sleep disorder due to occasional stress.

9. The method of claim 1, wherein gaboxadol is in an oral dose form selected from the group consisting of a solid oral dose form and a liquid oral dose form.

10. The method of claim 9, wherein the solid oral dose form is a tablet or a capsule.

11. The method of claim 1, wherein said human subject is selected from the group consisting of an elderly subject and an adult subject.

12. A method for treating primary insomnia in an adult or elderly human subject in need thereof comprising administering to said subject an oral dose form comprising 5 to 15 mg of gaboxadol per day, wherein the oral dose form is administered from about 1 hour before bedtime, up to bedtime.

13-27. (canceled)

28. A method for the preparation of a medicament for treating primary insomnia in an adult or elderly human subject comprising formulating 5 to 15 mg of gaboxadol in an oral dose form suitable for administration from about 1 hour before bedtime, up to bedtime.

29-41. (canceled)
